(19) United States
(12) Patent Application Publication
(10) Pub. No.: US 2024/0233089 A1
(43) Pub. Date: Jul. 11, 2024

(54) IMAGE PROCESSING METHOD AND IMAGE PROCESSING APPARATUS

(71) Applicant: SHIMADZU CORPORATION, Kyoto-shi, Kyoto (JP)

(72) Inventor: Ryuji SAWADA, Kyoto-shi, Kyoto (JP)

(73) Assignee: SHIMADZU CORPORATION, Kyoto-shi, Kyoto (JP)

(21) Appl. No.: 18/558,605

(22) PCT Filed: May 20, 2022
(86) PCT No.: PCT/JP2022/020948

§ 371 Date: Nov. 2, 2023

(30) Foreign Application Priority Data
Jul. 29, 2021 (JP) 2021-124707

(51) Int. Cl. G06T 5/50; G06T 5/20; G06T 5/70; G06V 10/60; G06V 20/69

(52) U.S. Cl. CPC G06T 5/50; G06T 5/20; G06T 5/70; G06V 10/60; G06V 20/695; G06T 2207/20032; G06T 2207/20221

(57) ABSTRACT

An image processing method according to this invention includes a step of converting pixel values of pixels in a cell image (31) into relative values; a step of acquiring a frequency-component extraction image (50) that extracts a predetermined frequency component from the converted cell image (40); a step to acquiring a first image (61a) by binarizing the converted cell image (40), and a second image (62) by binarizing the frequency-component extraction image (50); and a step of generating a binary image (70) of the cell image (31) by combining the first image (61) and the second image (62).

### TECHNICAL FIELD

The present invention relates to an image processing method and an image processing apparatus.

### BACKGROUND ART

Technologies of binarization applied to cell images are disclosed in the art. Such a technology is disclosed in Japanese Patent Laid-Open Publication No. JP 2014-18184, for example.

The above Japanese Patent Laid-Open Publication No. JP 2014-18184 discloses a technique of failure/no-failure evaluation of colonies of induced pluripotent stem cell based on differential filtered images of microscopic images using an optical microscope. In addition, the above Publication discloses that binarization is applied to the images to improve accuracy of image analysis.

### PRIOR ART

### Patent Document

Patent Document 1: Japanese Patent Laid-Open Publication No. JP 2014-18184

### SUMMARY OF THE INVENTION

### Problems to be Solved by the Invention

The binarization of cell images is used to separate cell areas from background areas, and to analyze morphological features such as cell size. However, the following problems arise in the binarization of cell images when areas of cells are accurately extracted.

Firstly, in a case which brightness is uneven in a cell image due to illumination variation in a microscope image, it becomes difficult to specify a threshold to distinguish between a cell area and a background area.

Secondly, the brightness level of an inner area of a cell and a subcellular structure of a cell likely low in a cell image. If the aforementioned uneven brightness is superimposed on such a low-brightness area of a cell, it is difficult to accurately extract the low-brightness area of the cell by using binarization. For example, a low-brightness part of an inner area of a cell is seen as a hole formed inside the cell in a binary image. Such subcellular structures include a pseudopodium, which is a temporary projection of a cell main body. When binarization is applied to a cell that has a pseudopodium, a low brightness part of the pseudopodium cannot be distinguished from a background so that the pseudopodium can be separated from the cell main body in a binary image. For example, in a case in which a size of a cell is calculated, the size of the cell area after binarization will be smaller (by a size corresponding to the separated pseudopodium, which is excluded from the size calculation) as compared to an actual size of the entire cell including the pseudopodium.

For this reason, it is desirable to reduce such an adverse effect of uneven brightness in particular to microscopic images, and to accurately extract a low-brightness area of a cell including a subcellular structure of the cell in binarization of a cell image.

The present invention is intended to solve the above problems, and one object of the present invention is to provide an image processing method and an image processing apparatus capable of reducing an adverse effect of uneven brightness on a cell image and accurately extracting a low-brightness area of a cell including a subcellular structure of the cell in binarization of a cell image.

### Means for Solving the Problems

In order to attain the aforementioned object, an image processing method according to a first aspect of the present invention is an image processing method for binarization of a multi-value cell image, the method including a step of extracting a background brightness distribution in the cell image; a step of converting pixel values of pixels in the cell image into relative values relative to the background brightness distribution; a step of acquiring a frequency-component extraction image that extracts a predetermined frequency component corresponding to a subcellular structure of a cell from the converted cell image; a step to acquiring a first image by binarizing the converted cell image, and a second image by binarizing the frequency-component extraction image; and a step of generating a binary image of the cell image by combining the first image and the second image.

An image processing apparatus according to a second aspect of the present invention includes an image acquirer that is configured to acquire a multi-value cell image; a background extractor that is configured to extract a background brightness distribution in the cell image; a relativizer that is configured to convert pixel values of pixels in the cell image into relative values relative to the background brightness distribution; a frequency component extractor that is configured to acquire a frequency-component extraction image that extracts a predetermined frequency component corresponding to a subcellular structure of a cell from the converted cell image; a binarizer that is configured to acquire a first image by binarizing the converted cell image by using binarization, and a second image by binarizing the frequency-component extraction image by using binarization; and a combiner that is configured to generate a binary image of the cell image by combining the first image and the second image.

### Effect of the Invention

In the image processing method according to the first aspect and the image processing apparatus according to the second aspect, because background brightness distribution in the cell image is extracted, and pixel values of pixels in the cell image are converted into relative values relative to the background brightness distribution, the converted pixel values in the cell image represent degrees of difference from the brightness levels of the background. Accordingly, even if brightness is uneven in the cell image, because binarization can be applied based on the degrees of difference from the brightness levels of the background, it is possible to reduce adverse effects of uneven brightness. Because binarization can be applied to the converted cell image that is represented by the relative values, the first image can be accurately extracted even in a low-brightness area of the cell that is close to the background brightness level. Also, because binarization is applied to a frequency-component extraction image that extracts a predetermined frequency component corresponding to a subcellular structure of the cell, the second image that accurately extracts the subcellular structure of the cell can be acquired. Also, because the first image and the second image are combined so that the second image, which extracts the subcellular structure, complements the first image, which extracts the main morphology of the cell, it is possible to acquire a binary image that accurately extracts the entire morphology of the cell including the low brightness area in the cell. Consequently, it is possible to reduce an adverse effect of uneven brightness on a cell image, and to accurately extract a low-brightness area of a cell including a subcellular structure of the cell in binarization of a cell image.

### BRIEF DESCRIPTION OF THE DRAWINGS

FIG. 1 is a block diagram an image processing system including an image processing apparatus according to an embodiment.

FIG. 2(A) is a view of an exemplary cell image, FIG. 2(B) is a view of an exemplary binary image of the cell image, FIG. 2(C) is an enlarged view of an exemplary of the cell image, and FIG. 2(D) is a binary image corresponding to the enlarged view.

FIG. 3 is a functional block diagram functions of processors of the image processing apparatus.

FIG. 4 is a flowchart illustrating processes of the image processing apparatus according to this embodiment.

FIG. 5 is a diagram illustrating details of preprocessing.

FIG. 6 is a diagram illustrating details of the extraction of background brightness distribution and conversion of the background brightness distribution to relative values.

FIG. 7 is a diagram illustrating brightness distributions of a cell image and a background image.

FIG. 8 is a diagram illustrating a brightness distributions of a normalized image.

FIG. 9 is a diagram illustrating details of generation of a frequency-component extraction image.

FIG. 10 is a diagram illustrating a frequency band included in the frequency-component extraction image.

FIG. 11 is a diagram illustrating selection of a set of parameters for smoothing.

FIG. 12 is diagram a illustrating details of binarization.

FIG. 13 is a diagram illustrating details of combination of a first image and a second image.

FIG. 14 is a diagram illustrating details of post-processing.

FIG. 15(A) is a view showing a binary image after post-processing according to this embodiment, and FIG. 15(B) is a view showing a binary image of a comparative example.

FIG. 16 is a block diagram showing an image processing apparatus according to a modified embodiment.

### MODES FOR CARRYING OUT THE INVENTION

Embodiments embodying the present invention will be described with reference to the drawings.

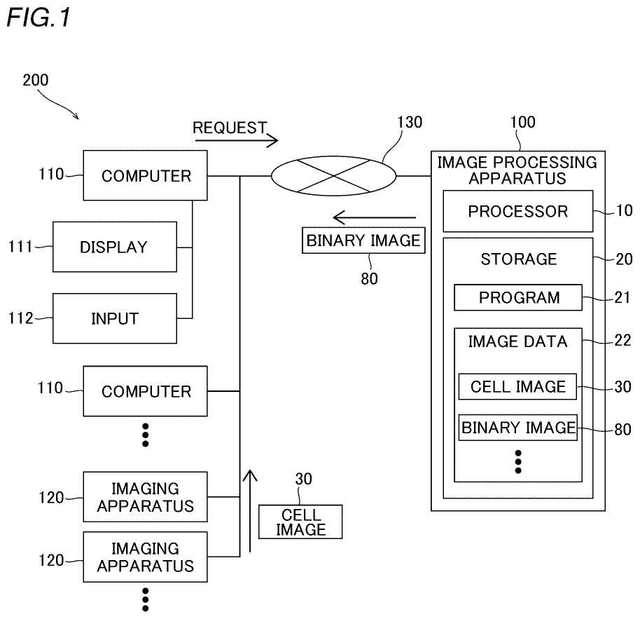

The following description describes a configuration of an image processing system 200 including an image processing apparatus 100, and an image processing method according to an embodiment with reference to FIGS. 1 to 15.

### (Image Processing System)

The image processing system 200 shown in FIG. 1 is a single system that integrally allows users who culture cell, etc. to capture cell images 30, to apply image processing to the cell images 30, and to peruse images to which image processing is applied.

### (Outline of Image Processing System)

The image processing system 200 includes the image processing apparatus 100, a computer 110, and an imaging apparatus 120.

The image processing system 200 shown in FIG. 1 is illustratively constructed of a client-server model. The computer 110 serves as a client terminal in the image processing system 200. The image processing apparatus 100 serves as a server in the image processing system 200. The image processing apparatus 100, the computer 110, and the imaging apparatus 120 are connected through a network 130, and can communicate with each other through the network. The image processing apparatus 100 performs various types of information processing in response to requests (processing requests) from the computer 110 operated by users. The image processing apparatus 100 is configured to apply image processing to the cell images 30 in response to request and to provide the computer 110 with images to which image processing is applied. A graphical user interface (GUI) is displayed on a display 111 of the computer 110, and is configured to accept instructions provided to the image processing apparatus 100 and to display images to which image processing is applied by the image processing apparatus 100.

The network k 130 connects the image processing apparatus 100, the computer 110, and the imaging apparatus 120 to each other so that they can communicate with each other. The network 130 can be a local area network (LAN) configured in the facility, for example. The network 130 can be the Internet, for example. In a case in which the network 130 is the Internet, the image processing system 200 can be a system configured in a cloud computing form.

The computer 110 is a so-called personal computer including a processor and a storage. The display 111 and an input 112 are connected to the computer 110. For example, the display 111 is a liquid crystal display. The display 111 may be an electro-luminescence display, a projector or a head-mounted display. For example, the input 112 is an input device including a computer mouse and a key-board. The input 112 may be a touch panel. The image processing system 200 can includes one or more computers 110.

The imaging apparatus 120 is configured to generate cell images 30. The imaging apparatus 120 can send the generated cell images 30 to the computer 110 and/or the image processing apparatus 100 through the network 130. The imaging apparatus 120 is configured to capture microscopic images of cells. The imaging apparatus 120 can use imaging methods such as bright-field observation, dark-field observation, phase contrast observation, and differential interference observation, etc. for imaging. One or more of the imaging apparatuses 120 are used in accordance with imaging methods. The image processing system 200 can include one or more imaging apparatuses 120.

The image processing apparatus 100 includes a processor 10, such as CPU (Central Processing Unit), FPGA (Field-Programmable Gate Array), and ASIC (Application Specific Integrated Circuit). The image processing apparatus 100 is configured to perform arithmetic processing by using the processor 10 by executing a predetermined program 21.

The image processing apparatus 100 includes a storage 20. The storage 20 includes a non-volatile storage device. For example, the non-volatile storage device is a hard disk drives, solid state drives, etc. The storage 20 is configured to store various programs 21 to be executed by the processor 10. The storage 20 is configured to store image data 22. The image data 22 includes the cell images 30 captured by the imaging apparatus 120, and various images generated by applying image processing to the cell images 30 (binary images 80). Among functions of image processing that can be performed by the image processing apparatus 100, binarization of a cell image 30 is described in this embodiment.

The image processing apparatus 100 apply binarization to the cell image 30 in response to a request from the computer 110. As a result of image processing, the image processing apparatus 100 can generate the binary image 80 of the cell image 30. The image processing apparatus 100 sends the generated binary image 80 to the computer 110. The computer 110 receives the information, and displays the binary image 80 on display 111.

### <Cell Image>

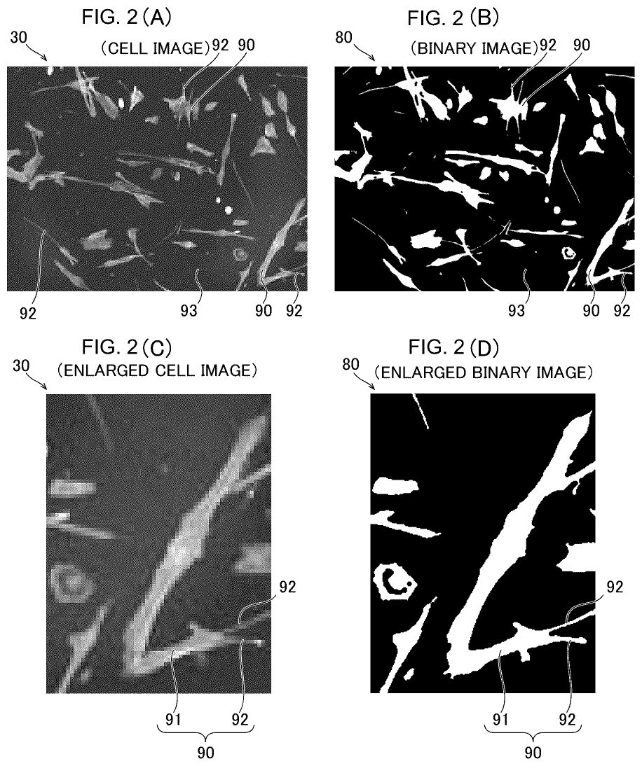

As shown in FIG. 2, a cell image 30 is a microscopic image of cultured cells cultured by using cell culture tools, for example. Although a type of the cell image 30 is not specifically limited, the cell image 30 is a fluorescent stained images of cells in this embodiment, for example. The cell image 30 is a multi-value image (multi-level image), and a color image.

The cell image 30 includes images of cells 90 (cellular images), and a background 93. The cell 90 in the cell image 30 shown in FIG. 2(A) includes a main body 91 and a subcellular structure 92. The exemplary subcellular structure 92 shown in FIG. 2 is a pseudopodium that is a protrusion of cytoplasm from the main body 91, more specifically a filipodia that protrudes in a thread shape (filament-shape) from the main body 91. In FIGS. 2(A) and 2(C), areas of the subcellular structures 92 and inner areas of the cell main bodies 91 are likely to include a low brightness area, which has relatively low brightness (low pixel value) in the cells 90 (they are likely to be dark in the cell image 30).

As shown in FIGS. 2(B) and 2(D), binarization is processing for converting a pixel that has a pixel value not smaller than a binarization threshold to “1 (white)”, and a pixel that has a pixel value smaller than the binarization threshold to “0 (black)”. In the binarization of the cell image 30, the cell image 30 is binarized so that the images of the cells 90 (cellular images) are extracted as white areas, and the background 93 other than the cells 90 are represented by a black area. The binarization threshold is set to a value that can distinguish between a range of pixel value to which the cellular image belongs, and a range of pixel value to which the background 93 belongs.

For this reason, if the brightness distribution of the background 93 is uneven so that relatively high and low brightness areas are included in the background 93, because difference between pixel values of the low brightness area in the cell 90 and pixel values of the high brightness area in the background 93 becomes small, it is difficult to extract the low-brightness areas (to convert the low-brightness areas to white areas) in the binarization.

The image processing apparatus 100 according to this embodiment can generate binary images 80 that accurately extract low brightness areas in the cells 90, even when a brightness distribution of the background 93 is uneven in the cell images 30 as shown in FIGS. 2(B) and 2(D). The following description describes the image processing apparatus 100 in detail.

### (Detailed Configuration of Image Processing Apparatus)

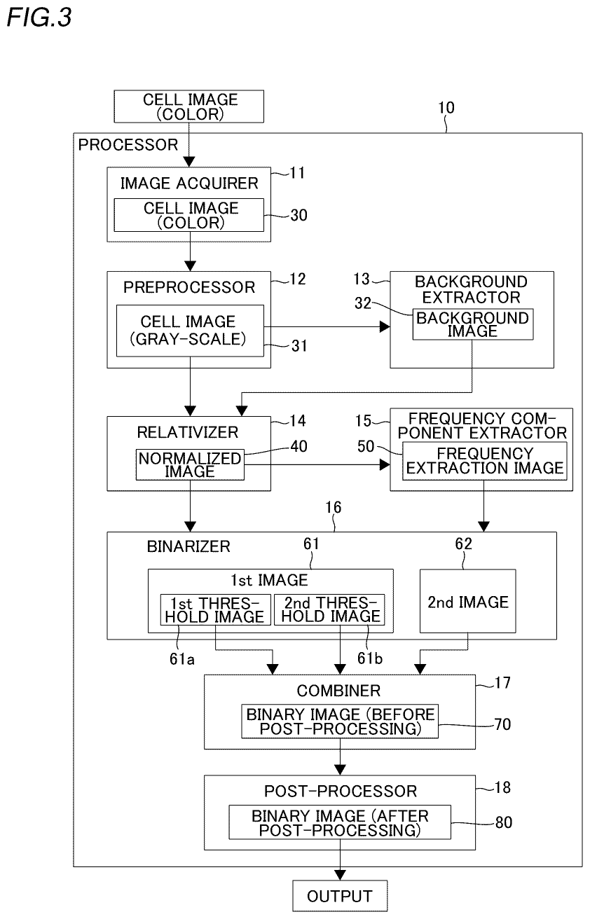

FIG. 3 is a block diagram showing a configuration of the image processing apparatus 100 relating to the binarization, and an outline of the image processing.

The processor 10 of the image processing apparatus 100 includes an image acquirer 11, a preprocessor 12, a background extractor 13, a relativizer 14, a frequency component extractor 15, a binarizer 16, a combiner 17, and a post-processor 18 as functional blocks. In other words, the processor 10 is configured to execute the program 21 stored in the storage 20 to serve as the image acquirer 11, the preprocessor 12, the background extractor 13, the relativizer 14, the frequency component extractor 15, the binarizer 16, the combiner 17, and the post-processor 18.

The image acquirer 11 has a function of acquiring cell images 30. The image acquirer 11 is configured to read a cell image 30 stored in the storage 20 (see FIG. 1) and to acquire the cell image 30 to which the binarization is applied. The image acquirer 11 may acquire a cell image 30 sent from the imaging apparatus 120 or the computer 110 through the network 130 (see FIG. 1). The image acquirer 11 can provide the acquired cell image 30 to the preprocessor 12.

The preprocessor 12 is configured to perform preprocessing for the binarization. Specifically, the preprocessor 12 is configured to convert a color cell image 30 to a gray-scale cell image 31. A gray-scale image is a monochrome multi-level image (without color information). Pixel values of pixels in the cell image 31 represent brightness of the pixels (brightness of image). The preprocessor 12 can provide the gray-scale cell image 31 to the background extractor 13 and the relativizer 14.

The background extractor 13 has a function of extracting a background brightness distribution in the cell image 31. The background brightness distribution is a brightness distribution of ambient light (illumination light) in the cell image 31. Ideally, the background brightness distribution is even in the entire image, but practically, illumination intensity variation makes the background brightness distribution uneven in the cell images 31. Such background brightness distributions of cell images 31 will be different from each other because of exposure time variation etc. The background extractor 13 is configured to generate a background image 32 representing the background brightness distribution in the cell image 31. The background image 32 represents the background brightness distribution in the cell image 31 for every pixel of the cell image 31.

Here, a pixel value of each pixel in the cell image 31 can be considered as the sum of a cellular image component and a background brightness component. The cellular image components are image components of light signals containing image information of the cells 90 (see FIG. 2). The background brightness components are image components of the background light that is inevitably observed under environments for capturing images of the cell 90. From this viewpoint, the background brightness components of all pixels, i.e., the background brightness distribution can be obtained by removing the cellular image components is removed from all pixels of the cell image 31.

In this embodiment, the background extractor 13 is configured to generate the background image 32 representing the background brightness distribution by filtering the cell image 31 to remove the cells 90 in the cell image 31. The filtering the cell image to remove the cells 90 is median filter for a kernel size corresponding to the sizes of the cells 90 in the cell image 31. Median filtering is a process of replacing a pixel value corresponding to a target pixel at a center of a kernel with a median pixel value of surrounding pixels other than the target pixel. The background extractor 13 can provide the generated background image 32 to the relativizer 14. The background Image 32 is an example of a “background brightness distribution” in the claims.

The relativizer 14 is configured to convert a pixel value of each pixel in the cell image 31 to a value relative to the background brightness distribution. The relativizer 14 is configured to generate a normalized image 40 based on the background image 32 by converting pixel values of all pixels in the cell image 31 into relative values. As will be discussed later, the pixel value of each pixel in the normalized image 40 represents a ratio of brightness to background brightness of the pixel. The relativizer 14 can provide the generated normalized image 40 to the frequency component extractor 15 and the binarizer 16.

The frequency component extractor 15 is configured to extracts a predetermined frequency component corresponding to the subcellular structures 92 (see FIG. 2) of the cells 90, and to acquire a frequency-component extraction image 50 from the normalized image 40. Focusing attention on spatial frequencies of the image, the subcellular structures 92 of the cells 90 corresponds to a high-frequency component higher than a frequency of the background 93, which is a low frequency component, and can be clearly distinguished from the background 93. Accordingly, the frequency component extractor 15 can exclude the background 93 to generate frequency-component extraction image 50, which extracts the subcellular structures 92, by extracting a predetermined frequency component corresponding to the subcellular structures 92 from the normalized image 40. The frequency component extractor 15 can provide the generated frequency-component extraction image 50 to the binarizer 16.

The binarizer 16 is configure to binarize the normalized image 40 and the frequency-component extraction image 50. The binarizer 16 is configure to generate a first image 61 that is generated by binarizing the normalized image 40, and a second image 62 that is generated by binarizing the frequency-component extraction image 50. In this embodiment, the binarizer 16 is configure to generate a plurality of first images 61, which are a first threshold image 61a and a second threshold image 61b, by binarizing the normalized image 40 by using different binarization thresholds. The binarizer 16 can provide the first images 61 (first threshold image 61a and second threshold image 61b) and the second image 62 to the combiner 17.

The combiner 17 is configured to generate a binary image 70 of the cell image 31 by combining the first images 61, which are generated by binarizing the normalized image 40, and the second image 62, which is generated by binarizing the frequency-component extraction image 50. Main parts of the cells 90, except for the subcellular structures 92, are included in the first image 61. The subcellular structures 92 of the cells 90 are included in the second image 62, which is generated by binarizing the frequency-component extraction image 50. The binary image 70 that extracts both the main pars of the cells 90 and the subcellular structures 92 of the cells 90 can be acquired by combining these images. As described later, the first threshold image 61a is used for combination with the second image 62, and the second threshold image 61b is used for removal of noise from the second image 62. The combiner 17 can provide the binary image 70 of the cell image 31 to the post-processor 18.

The post-processor 18 is configured to apply processing such as image shaping and noise removal to the binary image 70, and to generate the binary image 80 after post-processing. The binary image 80 after post-processing is stored as the final binarization result of the provided cell image 30 in the storage 20. The binary image 80 is sent to the computer 110 in response to request, and is displayed on the display 111.

### (Image Processing Method)

The image processing method according to this embodiment is now described. The image processing method according to this embodiment is an image processing method for binarization of a multi-value cell image 31. The image processing method can be executed by the image processing apparatus 100 (processor 10).

The image processing method according to this embodiment has at least the following steps.
(1) A step of extracting a background brightness distribution (background image 32) in the cell image 30.
(2) A step of converting pixel values of pixels in the cell image 30 into relative values (normalized image 40) relative to the background brightness distribution.
(3) A step of acquiring a frequency-component extraction image 50 that extracts a predetermined frequency component corresponding to a subcellular structure 92 of the cell 90 from the converted cell image 31 (normalized image 40).
(4) A step of acquiring a first image 61 by binarizing the converted cell image 30, and a second image 62 by binarizing the frequency-component extraction image 50.
(5) A step of generating a binary image 70 of the cell image 31 by combining the first image 61 and the second image 62.

The background extractor 13 is configured to execute the step (1) of extracting a background brightness distribution in the cell image 31. The relativizer 14 is configured to execute the step (2) of converting pixel values of pixels in the cell image 31 into relative values relative to the background brightness distribution. The frequency component extractor 15 is configured to execute the step (3) of acquiring a frequency-component extraction image 50 that extracts a predetermined frequency component corresponding to a subcellular structure 92 of the cell 90 from the converted cell image 31. The binarizer 16 is configured to execute the step (4) of acquiring a first image 61 by binarizing the converted cell image 30, and a second image 62 by binarizing the frequency-component extraction image 50. The combiner 17 is configured to execute the step (5) of generating a binary image 70 of the cell image 31 by combining the first image 61 and the second image 62. The image processing method according to this embodiment further includes processing executed by the preprocessor 12 and processing executed by the post-processor 18.

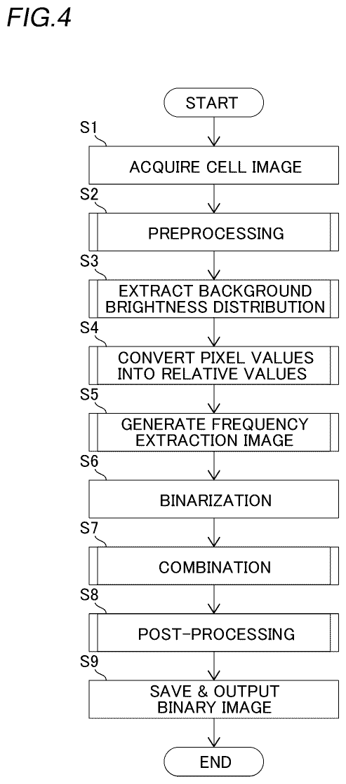

A processing flow executed by the image processing apparatus 100 is now described in detail with reference to FIGS. 4 to 15.

### <Image Acquisition>

In step S1, the image acquirer 11 (see FIG. 3) acquires a cell image 30 from the storage 20, the imaging apparatus 120 or the computer 110. The acquired cell image 30 is a multi-value and color image as discussed above.

### <Preprocessing>

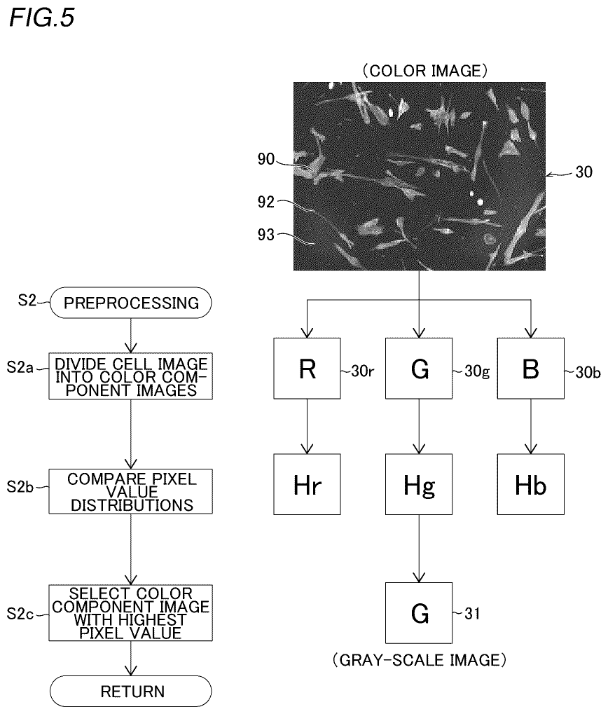

In step S2, the preprocessor 12 (see FIG. 3) executes preprocessing for binarization applied to the cell image 30 acquired in step S1. The preprocessing is described in detail with reference to FIG. 5.

In step S2a, the preprocessor 12 divides the cell image 30, which is a color image, into a plurality of color component images. The number of divided color component images corresponds to the number of color channels included in the cell image 30. In a case in which the color image is represented in RGB format, the cell image 30 is divided into three color component images, which are a red image 30r, a green image 30g, and a blue image 30b. Each color component image is a gray-scale image.

In step S2b, the preprocessor 12 acquires distributions of pixel values in the divided color component images (red image 30r, green image 30g, blue image 30b). The preprocessor 12, for example, creates histograms (histograms Hr, Hg, Hb) of pixel values of the color component images. The histogram represents the number of pixels, which are calculated for every pixel value, corresponding to each pixel value.

In step S2c, the preprocessor 12 compares the distributions of pixel values (histograms HR, Hg, HB) for the color component images, and selects the color component image having the highest pixel value. The preprocessor 12, for example, selects one color component image that has the highest average pixel value from the color component images. In an exemplary selection shown in FIG. 5, a green image 30g is selected as the color component image having the highest average pixel value. The preprocessor 12 provides the selected color component image (green image 30g) as a gray-scale cell image 31. As a result, the preprocessor 12 generates the gray-scale cell image 31 by dividing the color cell image 30 into a plurality of color component images and by selecting the color component image having the highest pixel value.

Typically, in a case in which a color image is converted into a gray-scale image, the average value of pixel values of each pixel in color component images is used as a pixel value of each pixel of a converted image. For this reason, brightness of a typical gray-scale image is average brightness of color component images. However, in fluorescent stained images of cells, brightness (pixel value) of a specific color component image that corresponds to a fluorescence wavelength is significantly higher, and brightness (pixel value) of the other color component images that do not contain the fluorescence wavelength is lower.

Accordingly, 4 typical gray-scale conversion is applied to the cell image 30, which is a fluorescent stained image, average brightness of the converted image reduces. To address this, averaged pixel values are not used but a color component image having the highest pixel value is used as a gray-scale cell image 31 to prevent such reduction of brightness of the gray-scale image.

### <Extraction of Background Brightness Distribution>

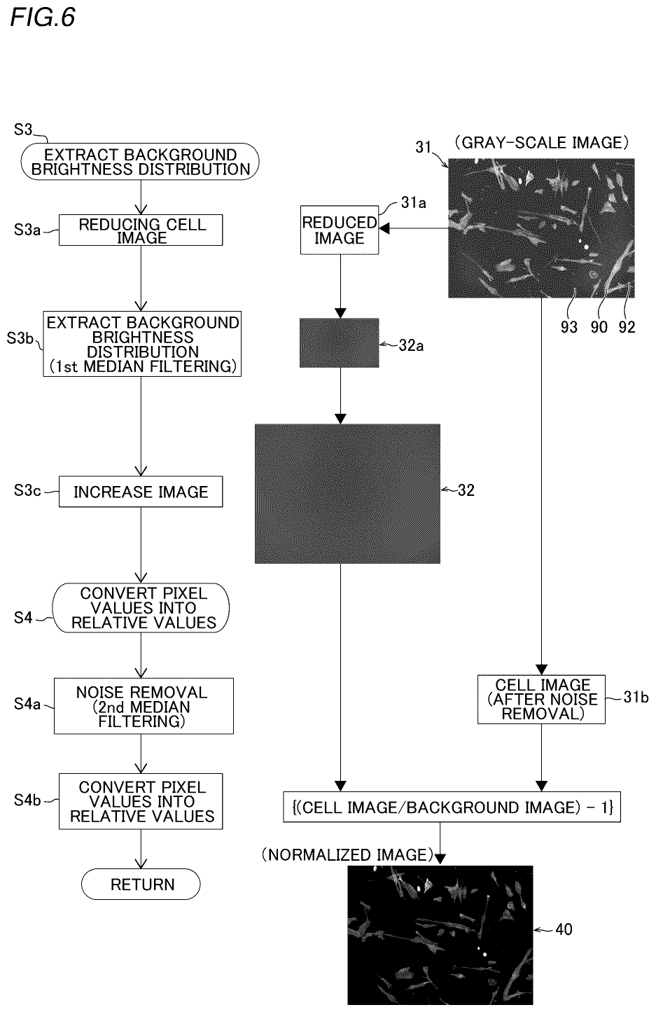

In step S3 of FIG. 4, the background extractor 13 extracts a background brightness distribution in the cell image 31 (the aforementioned step (1)). The preprocessing for extracting the background brightness distribution is described in detail with reference to FIG. 6.

The step S3 of extracting a background brightness distribution includes a step S3a of reducing the cell image 31, a step S3b of filtering the reduced cell image 31 to remove the cell 90 from the reduced cell image, and a step S3c of increasing the background image 32 after being subjected to the step of filtering the reduced cell image back to a size of the cell image before being subjected to the step of reducing the cell image.

In the step S3a, the background extractor 13 reduces the cell image 31 by a predetermined ratio. The reduction ratio is previously stored as setting information in the storage 20. Although not specifically limited, the reduction ratios can fall within a range from ½ to 1/10. The background extractor 13, for example, reduces the cell image 31 by ⅛. The reduced cell image 31 is referred to as a reduced image 31a.

In step S3b, the background extractor 13 applies first median filtering to remove the cells 90 from the reduced image 31a. A kernel size of the first median filtering is set to a value that is sufficiently remove the cells in the image and is enough small relative to periods of brightness variations in the background. The kernel size can be changed depending on imaging magnification of the cell image 30.

An exemplary kernel size of the first median filtering applied to the reduced image 31a is approximately 30 (pixels). For example, the kernel is a square pixel area of 30×30 pixels. Because the first median filtering is applied to the reduced image 31a reduced by a ⅛ scale, the first median filtering is equivalent to median filtering of an eight-times larger kernel size (240×240 pixels) that is applied to the cell image 31 before the reduction.

Although pixels that form images of the cells 90 (cellular images) have a higher pixel value than pixel values of pixels that belong to the background 93, when median filtering is applied using a sufficient size kernel, a median value is used for pixel values that belong to the background 93 surrounding the cells 90 so that pixel values of the pixels that form the cellular images are replaced by the pixel value of the background 93. As a result of the first median filtering, the cellular images are removed from the reduced image 31a so that a reduced background image 32a representing a background brightness distribution is generated.

In step S3c, the background extractor 13 enlarges the reduced background image 32a to return the reduced background image to its original image size. As discussed above, when the cell image 31 is reduced by ⅛ in step S3a, the background extractor 13 enlarges the reduced background image 32a by 8 times. As a result, a background image 32 having the same size as the cell image 31 is acquired.

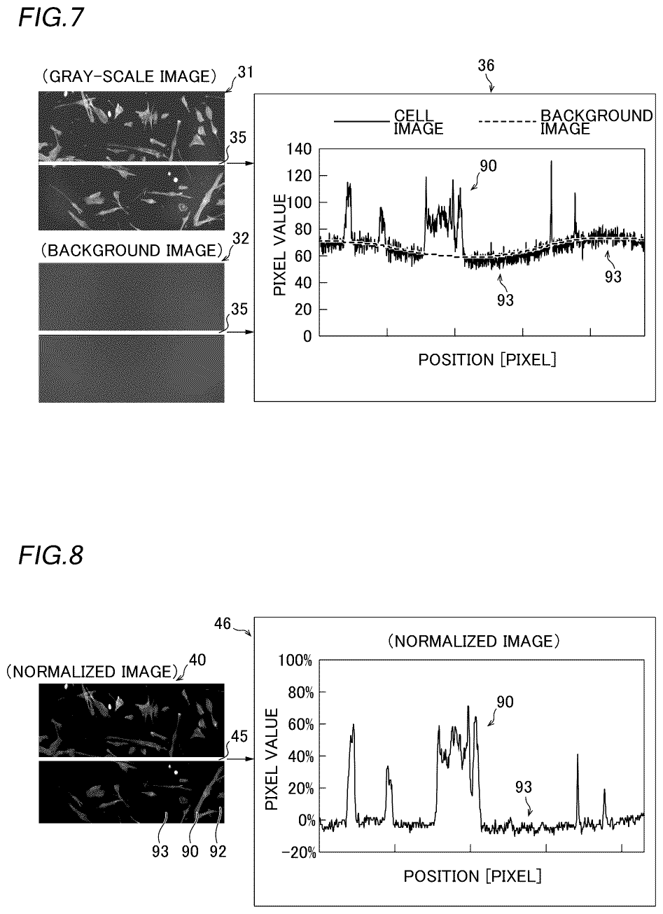

FIG. 7 is a graph 36 (brightness profile) of pixel values of pixels of the original cell image 31 and the acquired background image 32 along a common line 35. In the graph 36, a horizontal axis indicates a position (pixels along the line 35), and a vertical axis indicates pixel values.

In a pixel value distribution of the cell image 31 indicated by a solid line in the graph 36, areas of high pixel values that locally appear represent the cellular images, and the other baselines represent the background. The graph 36 shows that levels corresponding to background pixel values (brightness) are not constant but fluctuate depending on the position (the baseline is wavy). Such fluctuation of background brightness prevents extraction of low brightness areas in binarization. In the graph 36, the pixel value distribution of the background image 32 indicated by a dashed line shows that only a distribution of background brightness, which fluctuates depending on the position, is accurately extracted. Consequently, in step S3, the background image 32 is generated by the background extractor 13.

Although images are shown in different sizes in FIGS. 5 to 15 for ease of illustration, actual change of an image size is executed only in reduction of step S3a and enlargement of step S3c in this embodiment.

### <Relativization>

In step S4 of FIG. 4, the relativizer 14 (see FIG. 3) converts pixel values of pixels of the cell image 31 into relative values relative to the background brightness distribution (the aforementioned step (2)).

Specifically, as shown in FIG. 6, in step S4a, the relativizer 14 first applies filtering the cell image 31 to remove noise in the cell image 31 (second median filtering).

A kernel size of the second median filter is set to a size corresponding to small noise included in the cell image 31, and smaller than an effective size of the kernel of the first median filtering (kernel size converted to a size of the cell image 31 before reduction). An exemplary kernel size of the second median filtering is approximately a few pixels. For example, the kernel of the second median filtering is a square pixel area of 3×3 pixels. The cell image 31 after the second median filtering is referred to as a cell image 31b.

In step S4b, the relativizer 14 converts pixel values of pixels in the cell image 31b to relative values by dividing the pixel values of pixels in the cell image 31b by pixel values of pixels in the background image 32. Specifically, the relativizer 14 generates a normalized image 40 by using the following equation. Normalized image 40={(cell image 31b/background image 32)−1}×100(%). That is, a pixel value of each pixel in the normalized image 40 is a value that is obtained by dividing a pixel value of the same pixel in the cell image 31b by the pixel value of the same pixel in the background image 32 and by subtracting “1” from the divided value. A pixel value of each pixel in the normalized image 40 is represented by a percentage.

Because each pixel value of the cell image 31b are divided by each pixel value of the background image 32, the pixel value of each pixel in the normalized image 40 is a dimensionless quantity representing a ratio of brightness of an image signal to brightness of the background image 32. In other words, a pixel value of each pixel in the normalized image 40 represents a degree of difference from a brightness level of the same pixel in the background 93. The reason to subtract “1” after the dividing is to offset each pixel values to a level equal to the background brightness is the pixel value is 0%. Considering a pixel value of a pixel in the normalized image 40, if the pixel value=0% brightness of the pixel is a level equal to the background brightness of the same pixel, while if the pixel value=50% the pixel value is a level corresponding to 1.5 times the background brightness of the same pixel. Because a pixel value of each pixel in the cell image 31b is converted into a dimensionless quantity as a ratio of the pixel value to background brightness, the conversion into relative value in step S4b can be referred as “normalization”.

FIG. 8 is a graph 46 (profile) of pixel values of pixels of the normalized image 40 along a line 45. In the graph 46, a horizontal axis indicates a position (pixels along the line 45), and a vertical axis indicates pixel values (percentages). A position of the line 45 is the same as the line 35 indicated in the cell image 31 and the background image 32 shown in FIG. 7.

In the graph 46 showing the normalized image 40, levels of pixel values in the background (baseline) are roughly constant near 0% (in a range of −5% to 5%), which means that fluctuation of background brightness, which appears in the graph 36 of FIG. 7, is eliminated. For this reason, even if a fixed binarization threshold is applied to the entire image in the binarization, cell areas can be accurately distinguished from the background area. In other words, the brightness distribution of the background 93 (see FIG. 2) can be constant in the normalized image 40 so that low-brightness areas such as the subcellular structures 92 in the cell areas (see FIG. 2) and inner parts of the cell main bodies 91 (see FIG. 2) can be easily distinguished from the background 93.

### <Extraction of Frequency Component>

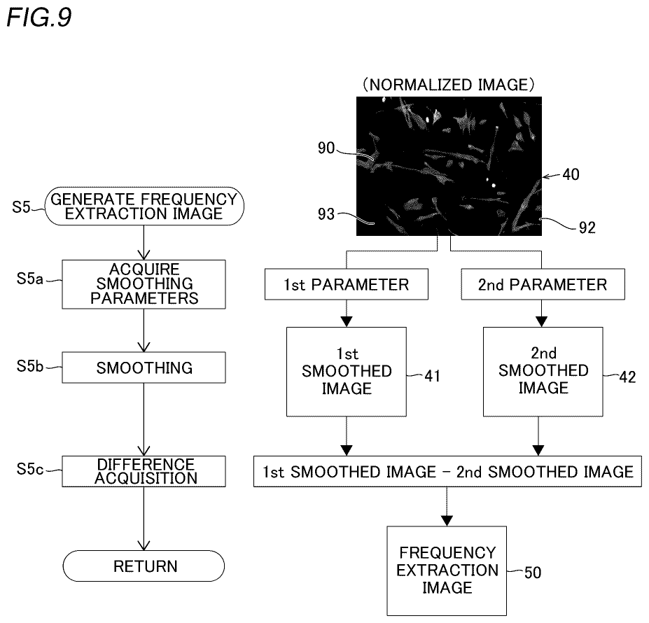

In step S5 of FIG. 4, the frequency component extractor 15 (see FIG. 3) is configured to execute the step (3) of acquiring the frequency-component extraction image 50 that extracts a predetermined frequency component corresponding to the subcellular structure 92 of the cell 90 from the normalized image 40 (relative-value-converted cell image 31). The preprocessing for acquiring the frequency-component extraction image 50 is described in detail with reference to FIG. 9.

The step S5 of acquiring a frequency-component extraction image 50 includes a step S5a of acquiring smoothing parameters (first and second parameters), a step S5b of generating a first smoothed image 41 and a second smoothed image 42 that have different frequency characteristics by smoothing the normalized image 40, and a step S5c of generating the frequency-component extraction image 50 based on difference between the first smoothed image 41 and the second smoothed image 42.

In step S5a, the frequency component extractor 15 acquires the smoothing parameters (first and second parameters) to generate the first smoothed image 41 and the second smoothed image 42. In this embodiment, the smoothing is Gaussian filtering, and parameters of the smoothing are standard deviations σ of the Gaussian filtering. A removed amount of high frequency components in the image increases (a larger amount of blurring is applied to the image) as a value of the parameter (standard deviation σ) becomes larger. The frequency component extractor 15 acquires the first and second parameters based on predetermined image processing conditions stored in the storage 20 (see FIG. 1). The second parameter is greater than the first parameter.

In step S5b, the frequency component extractor 15 acquires the first smoothed image 41 by applying Gaussian filtering to the normalized image 40 by using the first parameter. The frequency component extractor 15 acquires the second smoothed image 42 by applying Gaussian filtering to the normalized image 40 by using the second parameter.

In step S5c, the frequency component extractor 15 acquires the frequency-component extraction image 50 based on difference of the first smoothed image 41 from the second smoothed image 42. The difference between images is the subtraction of a pixel value of a pixel in one of the images from a pixel value of the same pixel in another the image.

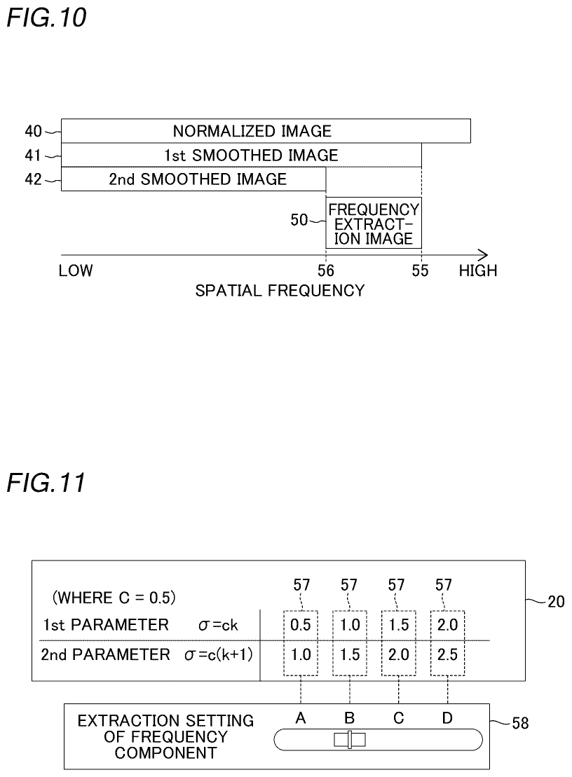

The frequency-component extraction image 50 is described with reference to a schematic diagram of FIG. 10. In comparison between spatial frequencies of the images, the first smoothed image 41 includes a frequency component that is obtained by excluding a range of the highest frequency side to a first frequency 55 in all frequency components included in the normalized image 40. The second smoothed image 42 includes a frequency component that is obtained by excluding a range of the highest frequency side to a second frequency 56 in the all frequency components included in the normalized image 40. As a result of difference between the smoothing parameters, the second frequency 56 is lower than the first frequency 55. For this reason, based on the difference of the first smoothed image 41 from the second smoothed image 42, it is possible to acquire an image including a frequency component corresponding to a frequency band between the first frequency 55 and the second frequency 56 (frequency-component extraction image 50).

Although the first frequency 55 and the second frequency 56 are intended to represent remove particular spatial frequencies for ease of illustration, because degrees of removal of high-frequency components are represented by Gaussian distributions in practical smoothing by Gaussian filtering, a higher frequency component cannot be completely removed from a particular spatial frequency (first frequency 55 or second frequency 56) that defines a frequency band dissimilar to the exemplary removal shown in FIG. 10. As a spatial frequency to be removed increases, a ratio of high frequency component gradually decreases in a smoothed image (the image is blurred).

As shown in FIG. 10, the frequency-component extraction image 50 is an extraction of image elements of a particular frequency band. The frequency band to be extracted is determined by the combination of the smoothing parameters (first and second parameters) for generation of the first smoothed image 41 and the second smoothed image 42. For this reason, the frequency-component extraction image 50 that selectively extracts the subcellular structures 92 included in the cell image 31 can be acquired by adjusting a frequency band to a frequency band including the subcellular structures 92 of the cells (see FIG. 2).

To achieve this, the smoothing parameters (first and second parameters) that can extract the frequency band including the subcellular structures 92 (filipodia) are selected for the cell image 31.

As shown in FIG. 11, parameter sets 57 of the first parameter and the second parameter are previously stored in the storage 20. In FIG. 11, the first parameter and the second parameter are represented by σ1=ck and σ2=c (k+1), respectively. c is a coefficient that defines a numerical interval between parameter sets 57. k is a variable that identifies each parameter set 57. In a case of c=0.5, a set 57 of k=1 includes σ1=0.5, and σ2=1.0. A set 57 of k=2 includes σ1=1.0, and σ2=1.5. Four parameter sets 57 are shown in FIG. 11.

A user can select one of settings A, B, C, and D corresponding to the variable k=1, 2, 3, and 4, respectively, for example, by operating an input 112 on a GUI 58 displayed on the display 111 (see FIG. 1). A selecting instruction accepted by the input is transmitted from the computer 110 to the image processing apparatus 100 through the network 130. As a result, in the aforementioned step S5a (see FIG. 9), the processor 10 (frequency component extractor 15) selects a parameter set 57, which includes a first parameter for generating the first smoothed image 41 and a second parameter for generating the second smoothed image 42, from predetermined parameter sets 57, which are stored in the storage 20, in accordance with the selecting instruction operated by the user. Consequently, the parameter set 57 corresponding to a frequency band to which the subcellular structures 92 in the cell image 30 belong is obtained in accordance with the user's intention.

### <Binarization>

The binarizer 16 (see FIG. 3) is configured to execute a process (the aforementioned step (4)) of acquiring a first image 61 by binarizing the normalized image 40 (converted cell image 31, and a second image 62 by binarizing the frequency-component extraction image 50 in step S6 in FIG. 4.

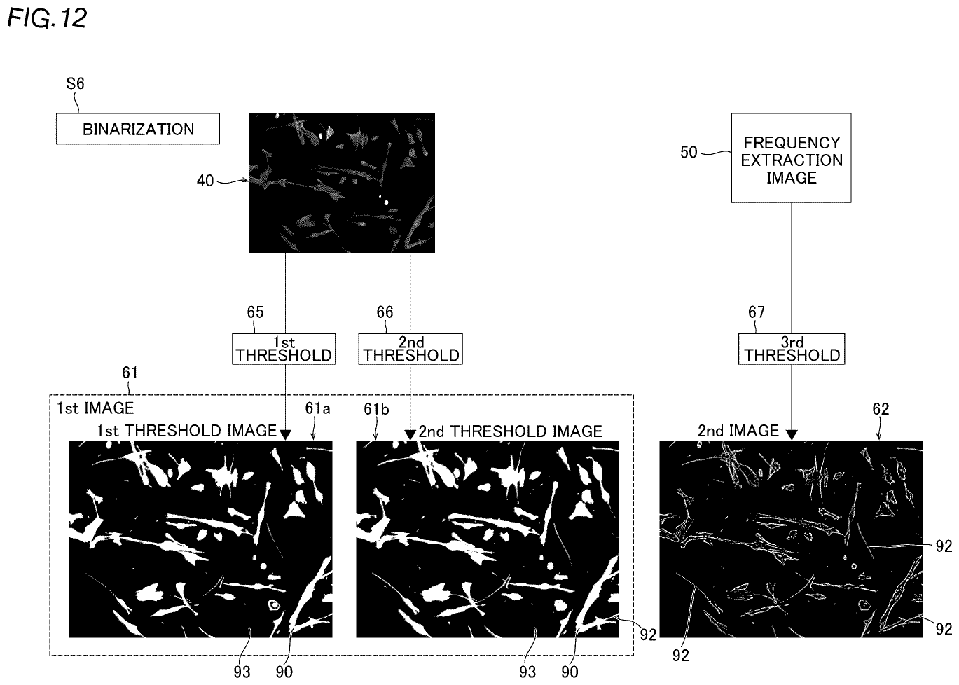

The binarization is described in detail with reference to FIG. 12. The binarizer 16 is configure to binarize the normalized image 40 generated in step S4 and the frequency-component extraction image 50 generated in step S5. The binarization in this embodiment is simple binarization that uses one binarization threshold and binarizes the entire image.

The binarizer 16 is configure to binarize the normalized image 40 by using a first threshold 65 and a second threshold 66 smaller than the first threshold 65. Correspondingly, the first threshold image 61a acquired by binarizing the cell image 31 by using the first threshold 65, and the second threshold image 61b acquired by binarizing the cell image 31 by using the second threshold 66 smaller than the first threshold 65 are included as the first image 61. In addition, the binarizer 16 is configure to generate the second image 62 by binarizing the frequency-component extraction image 50 by using a third threshold 67.

Because the second threshold 66 is set to a value lower than the first threshold 65, pixels that have lower brightness (lower pixel values) in the normalized image 40 are extracted as white areas in the second threshold image 61b. In the graph 46 of FIG. 8 showing a distribution of pixel values of the normalized image 40, the baseline corresponding to the background 93 is roughly constant near 0% (in a range of −5% to 5%). Accordingly, the second threshold is 10% and the first threshold is 20% as exemplary binarization thresholds. In this case, in the first threshold image 61a (see FIG. 12) acquired by binarizing the cell image by using the first threshold 65, which is set sufficiently higher relative to an upper limit of the baseline (5%), it is possible to sufficiently reduce a possibility that noise, which is not cells, is extracted as the white area (cellular image). Because the second threshold 66 is set close to the upper limit of the baseline (5%) as compared to the first threshold 65, it is possible to accurately extract areas that have relatively low brightness (low pixel value) in the cells 90 such as the subcellular structures 92. For this reason, there is a relatively higher possibility that noise and the background are extracted as the white areas (cellular images) in the second threshold image 61b (see FIG. 12) as compared to the first threshold image 61a.

Because the frequency-component extraction image 50 is an image that extracts image elements belonging to a frequency band including the subcellular structures 92 of the cells 90, the frequency-component extraction image does not include image elements in a low frequency band to which the background 93 belongs, and as a result the background 93 may not be extracted as the white area even if the third threshold 67 is set low. For this reason, the third threshold is 1% as exemplary binarization threshold for the frequency-component extraction image 50. In other words, pixels that have a pixel value greater than zero are extracted as cell areas (white areas) in the frequency-component extraction image 50. Accordingly, as shown in FIG. 12, image elements corresponding to the cell outlines including the subcellular structures 92 of the cells 90 can be extracted as white areas (cellular images) in the second image 62. Consequently, in this embodiment, the third threshold 67 is set lower than the binarization thresholds (first threshold 65, second threshold 66) for normalized image 40.

### <Combination>

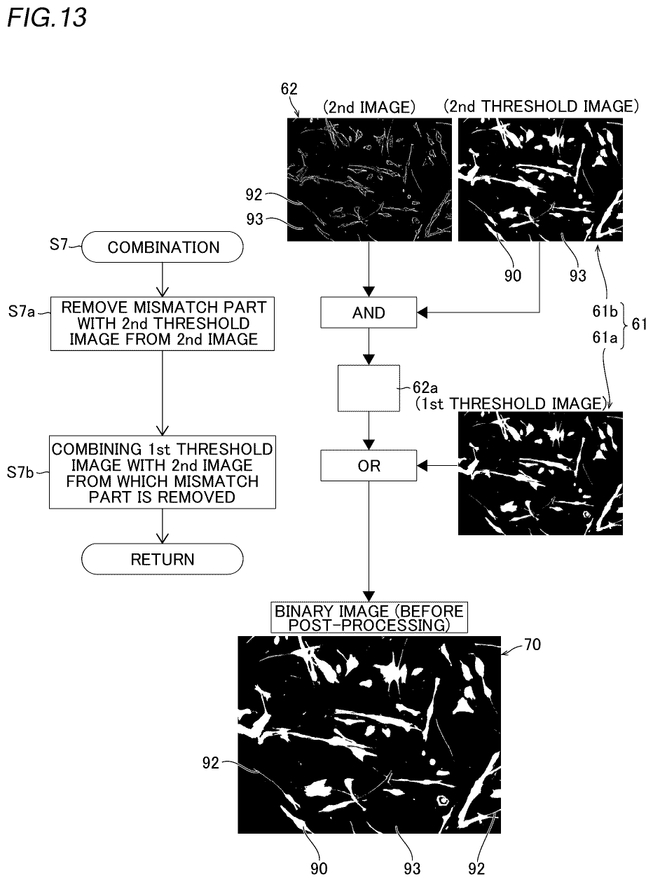

The combiner 17 is configured to execute combination (the aforementioned step (5)) of generating a binary image 70 of the cell image 31 by combining the first images 61 (binarized cell image 31) and the second image 62 (binarized frequency-component extraction image 50) in step S7 of FIG. 4. The combination is described in detail with reference to FIG. 13.

In this embodiment, the step of generating a binary image 70 of the cell image 31 includes a step S7a of removing a mismatch part of the second image 62 that does not match with the second threshold image 61b from the second image 62, and a step S7b of combining the first threshold image 61a with the second image 62a from which the mismatch part, which does not match with the second threshold image 61b, is removed.

In step S7a, the combiner 17 implements logical conjunction (AND) between the second threshold image 61b and the second image 62. In other words, the combiner 17 compares one pixel in the second threshold image 61b with the same pixel in the second image 62, and sets the pixel to “1 (white)” if the set of pixel values of the pixel of them is “1:1” or sets the pixel to “0 (black)” if the set of pixel values of the pixel of them is “0:0”. In a case of mismatch sets (1:0, 0:1) in which pixel values of the pixel of them do not match with each other, the pixel is set to “0 (black)”. Here, (1:1) means “pixel value of second threshold image 61b:pixel value of second threshold image 62”. As a result, pixel values of the mismatch part of the second image 62 that does not match with the second threshold image 61b are converted to “0” (black color) from the second image 62 so that the mismatch part between the second threshold image 61b and the second image 62 is removed.

Image elements of outlines of the cells in the second image 62 captured commonly to the second threshold image 61b can be left in the white area, while image elements such as noise captured only in one of the second image 62 and the second threshold image 61b can be removed by implementing logical conjunction between the second threshold image 61b and the second image 62. From this viewpoint, step S7a is a process for removing noise.

For example, if the original cell image 30 is not a high quality image and includes a relatively large number of noise factors, step S7a can effectively remove noise from the second image 62. On the other hand, if the original cell image 30 is a high quality image and includes few noise factors, step S7a may not be performed because there is little change in the second image 62 after implementation of the logical conjunction between the second threshold image 61b and the second image 62. Hereinafter, the second image 62 from which the mismatch part between the second threshold image 61b and the second image 62 is removed in step S7a is referred to as a “second image 62a”.

Subsequently, in step S7b, the combiner 17 implements logical disjunction between the first threshold image 61a and the second image 62a whereby combining the first threshold image 61a and the second image 62a.

In other words, the combiner 17 compares one pixel in the first threshold image 61a with the same pixel in the second image 62a, and sets the pixel to “1 (white)” if any of pixel values of the pixel of them is “1 (white)” (in a case of any of sets 1:1, 1:0, and 0:1), or sets the pixel to “0 (black)” if both pixel values of the pixel of them are “0” (black) (in a case of 0:0). The combiner 17 generates the binary image 70 by the combination (logical OR operation),

The binary image 70 that complements the cellular image extracted in the first threshold image 61a by adding the subcellular structure 92, which cannot be easily extracted, can be acquired by implementation of logical disjunction between the first threshold image 61a and the second image 62a.

### <Post-Processing>

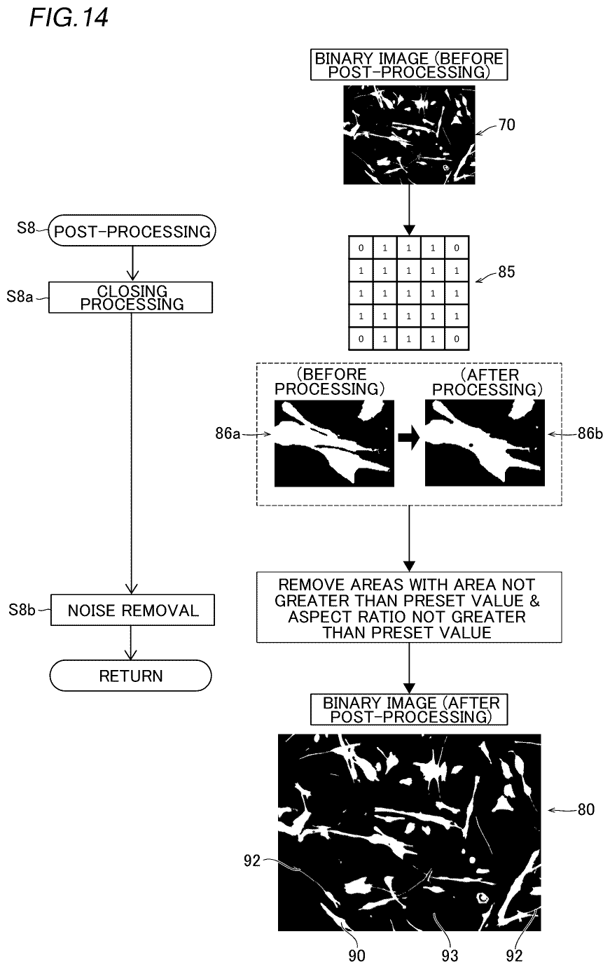

The post-processor 18 is configured to apply processing such as image shaping and noise removal to the binary image 70 in step S8 of FIG. 4. The post-processing is described in detail with reference to FIG. 14.

The post-processor 18 is configured to apply image shaping to the binary image 70 (before post-processing) that is provided from the combiner 17 in step S8a. Image shaping is a process for shaping a cellular image to interpolate local defects in the cellular image.

Specifically, the post-processor 18 applies closing processing to the binary image 70. The closing processing is a process for expanding white areas in the image and then contracting the white areas. The closing processing can connect disconnected lines separated by a short distance and can fill holes (black areas) that locally exist in the cellular image without changing sizes of the cellular images (white areas).

For example, the post-processor 18 executes one expansion process and one contraction process for closing processing by using a kernel 85 shown in FIG. 14. The kernel 85 has a shape excluding four corners of rectangular area. As a result, it is possible to prevent that the cellular image after the shaping process based on the closing processing has an unnaturally cornered shape.

As shown in enlarged views 86a and 86b before and after the closing processing of FIG. 14, a thin notch and a thin hole, which exist in the enlarged view 86a before the closing processing, are filled in the enlarged view 86b after the closing processing.

Subsequently, the post-processor 18 applies noise removal to the binary image 70 after the image shaping in step S8b.

The noise removal in step S8b is a process for removing small spot noises that exist in the image. Specifically, the post-processor 18 removes white areas that have an area (number of pixels) not greater than a predetermined value and an aspect ratio not greater than a predetermined value (in other words, sets pixel values of these white areas to “0” (black)) in the white areas that exist in the image. The aspect ratio refers to a value of (long side/short side) of a minimum rectangle that encloses a target white area. For example, white areas that have an area not greater than 15 pixels and an aspect ratio not greater than 3.0 are replaced by black areas. As a result, the small spot noises, which exist in the binary image 70, can be removed.

As a result of the noise removal, the post-processor 18 can generate the binary image 80 after the post-processing. The binary image 80 after post-processing is provided as the final binarization result (processed image) of the cell image 30 provided to the image processing apparatus 100.

### <Storage and Output of Processed Image (Binary Image)>

In step S9 of FIG. 4, the processor 10 stores the binary image 80 after the post-processing provided from the post-processor 18 in the storage 20. The processor 10 also sends the binary image 80 after the post-processing to the computer 110 through the network 130. The computer 110 displays the binary image 80 as a processed result on the display 111.

In this embodiment, in addition to the binary image 80 after the post-processing, the images generated in steps S2 to S8 of the image processing are stored in the storage 20 as image data 22 (see FIG. 1). The processor 10 can send these images stored in the storage 20 to the computer 110 in response to request provided from the computer 110.

Accordingly, in a case in which a user wants to review the binary image 80 after the post-processing and to change the image processing conditions, the user can review the first smoothed image 41, the second smoothed image 42 and the frequency-component extraction image 50 and can determine whether settings of the frequency band (selection of smoothing parameters) to extract the subcellular structures 92 is appropriate or whether the binarization thresholds for the first threshold image 61a, the second threshold image 61b, and the second image 62 are appropriate whereby visually checking validity of the image processing conditions based on images in the processes.

Finally, the image processing method executed by the image processing apparatus 100 in this embodiment is completed.

### Operations of the Embodiment

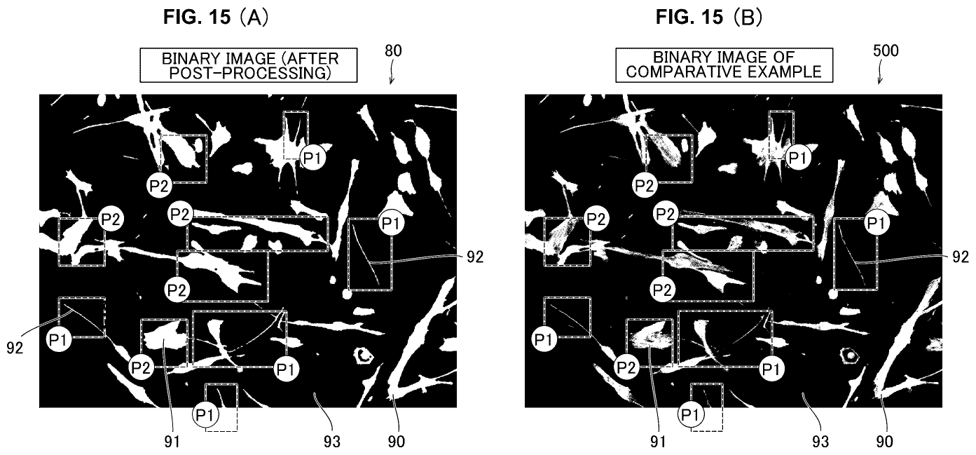

The image processing method according to this embodiment is now described with reference to FIG. 15. FIG. 15(A) is a view showing a binary image 80 after post-processing acquired from the cell image 30 by the image processing method according to this embodiment. FIG. 15(B) is a view showing a binary image 500 acquired from the same cell image 30 by a known binarization of a comparative example.

The binary image 500 in the comparative example is an image acquired by adaptive binarization. Adaptive binarization is known as a process that calculates binarization thresholds for small areas in the image, and separately applies the obtained binarization thresholds to the small areas so that an adverse effect of uneven brightness on the image can be reduced.

In comparison between the binary image 80 in this embodiment and the binary image 500 in the comparative example, in particular, in areas P1, filament-like subcellular structures (filipodia of cells) are broken/disconnected in the binary image 500 of the comparative example, but they are extracted as continuous filament-like part in the binary image 80 of this embodiment, and as a result extraction accuracy of the subcellular structure is improved. Also, in particular, in areas P2, due to superposition of uneven brightness and brightness variation of the cells themselves, relatively low brightness parts in inner areas of the cell main bodies 91 cannot be extracted in the binary image 500 of the comparative example, and as a result the images are rough. Contrary to this, in the binary image 80 of this embodiment, the inner areas of the main bodies 91 shown in the areas P2 can be extracted as uniform white areas.

In this embodiment, it is confirmed that the accuracy of extraction of subcellular structures can be improved and the adverse effect of uneven brightness can be reduced as compared to the binarization method (adaptive binarization), which is used to reduce the adverse effect of uneven brightness.

### Advantages of the Embodiment

In this embodiment, the following advantages are obtained.

That is, in the image processing method and the image processing apparatus 100 according to this embodiment, because background brightness distribution (background image 32) in the cell image 31 is extracted, and pixel values of pixels in the cell image 31 are converted into relative values relative to the background brightness distribution, the pixel values in the normalized image 40 after the conversion represent degrees of difference from the brightness levels of the background 93. Accordingly, even if brightness is uneven in the cell image 31, because binarization can be applied based on the degrees of difference from the brightness levels of the background, it is possible to reduce adverse effects of uneven brightness 93 on the cell image 31. Because binarization can be applied to the normalized image 40, the first image 61 can be accurately extracted even in a low-brightness area of the cell 90 that is close to the background brightness level. Also, because binarization is applied to a frequency-component extraction image 50 that extracts a predetermined frequency component corresponding to a subcellular structure 92 of the cell 90, the second image 62 that accurately extracts the subcellular structure 92 of the cell 90 can be acquired. Also, because the first image 61 and the second image 62 are combined so that the second image 62, which extracts the subcellular structure 92, complements the first image 61, which extracts the main morphology of the cell, it is possible to acquire a binary image 70 that accurately extracts the entire morphology of the cell 90 including the low brightness area in the cell 90. Consequently, it is possible to reduce an adverse effect of uneven brightness on a cell image, and to accurately extract a low-brightness area of a cell including a subcellular structure of the cell in binarization of a cell image.

In addition, additional advantages can be obtained by this embodiment added with configurations discussed below.

That is, in this embodiment, a background image 32 representing the background brightness distribution is generated by filtering the cell image 31 to remove the cell 90 in the cell image 31 in the step S3 of extracting a background brightness distribution. According to this configuration, because the background image 32, which is acquired by removing the cells 90 from the cell image 31, is generated, it is easily acquire a background brightness distribution (pixel values of pixels in the background) of the cell image 31 without complicated processing such as image analysis.

In this embodiment, the filtering the cell image to remove the cells 90 is median filter for a kernel size corresponding to the sizes of the cells 90 in the cell image 31. According to this configuration, the background image 32 representing the background brightness distribution can be easily acquired by applying median filtering on the cell image 31. Here, the median filtering can remove image elements (cells 90) while leaving a lower frequency component (background brightness distribution) larger than a kernel by setting a size of the kernel to an appropriate size corresponding to the image elements (cells 90 in this case) to be removed. As a result, the background brightness distribution of the cell image 31 can be accurately extracted.

In this embodiment, the step S3 of extracting a background brightness distribution includes a step S3a of reducing the cell image 31, a step S3b of filtering the reduced image 31a to remove the cell 90 from the reduced image, and a step S3c of increasing the background image after being subjected to the step of filtering the reduced cell image (reduced background image 32a) to remove the cell 90 back to a size of the cell image before being subjected to the step of reducing the cell image. According to this configuration, because filtering is applied to the reduced cell image (reduced image 31a), it is possible to reduce the kernel size in the filtering and to increase its effective kernel size when returning the reduced image size back to the original image size. As a result, an amount of calculation in the filtering can be effectively reduced by reducing the kernel size in the filtering.

In this embodiment, the pixel values of the pixels in the cell image 31 are converted into the relative values by dividing the pixel values of the pixels in the cell image by pixel values of pixels in the background image 32 in the step S4 of converting pixel values of pixels in the cell image into relative values relative to the background brightness distribution. According to this configuration, it is possible to easily convert a pixel value of each pixel in the cell image 31 to a relative value. Because a pixel value of each converted pixel represents a ratio of brightness of an image signal to the background brightness, it is possible to acquire a cell image (normalized image 40) independent on relative brightness of each pixel affected by uneven brightness. As a result, it is possible to reduce an adverse effect of pixel variation affected by uneven brightness also in binarization.

In this embodiment, the step S5 of acquiring a frequency-component extraction image 50 includes a step S5b of generating a first smoothed image 41 and a second smoothed image 42 that have different frequency characteristics by smoothing the normalized image 40, and a step S5c of generating the frequency-component extraction image 50 based on difference between the first smoothed image 41 and the second smoothed image 42. According to this configuration, a predetermine frequency component can be easily extracted from the normalized image 40 by smoothing the cell image 31 by using different parameters and by obtaining difference between the acquired first smoothed image 41 and second smoothed image 42. In the extraction of the frequency component, it is important to appropriately set a frequency range (frequency band) to be extracted depending on the subcellular structures 92 to be extracted. According to the aforementioned configuration, because the first smoothed image 41 and the second smoothed image 42, which are characterized by difference between the smoothing parameters, and the frequency-component extraction image 50, which is the difference between the first smoothed image 41 and the second smoothed image 42, are acquired, it is visually determine whether the subcellular structures 92 to be extracted are suitably extracted. Consequently, it is possible to easily optimize the smoothing parameters.

In this embodiment, the step S5 of acquiring a frequency-component extraction image 50 further includes a step S5a of selecting a parameter set 57, which includes a first parameter for generating the first smoothed image 41 and a second parameter for generating the second smoothed image 42, from sets of predetermined parameters 57. According to this configuration, a user can select one parameter set 57 suitable for a frequency band to which the subcellular structures 92 to be extracted belong from the plurality of parameter set 57 whereby determining the parameter set 57 for generating the first smoothed image 41 and the second smoothed image 42. As a result, even a user who does not have expert knowledge can easily determine smoothing parameters suitable for the subcellular structures 92 to be extracted.

In this embodiment, the smoothing is Gaussian filtering, and a parameter of the smoothing is a standard deviation of the Gaussian filtering. According to this configuration, a frequency components to be removed by Gaussian filtering can be easily adjusted by only one smoothing parameter. Also, because a high/low frequency component corresponds a large/small value of standard deviation, it is possible to easily determine smoothing parameters.

In this embodiment, the subcellular structure 92 of the cell 90 is a filipodia of the cell 90. In a microscopic image of cells 90, because the filipodias have a filament-like structure extending from the cell main body 91 so that pixel values of the filipodia are likely to be low (dark), the filipodia cannot be easily distinguished from the background 93 by simple binarization. For this reason, the binarization method according to this embodiment in which parts corresponding to filipodias are extracted based on the frequency-component extraction image 50 and then combined with the cell main body is particularly effective at generating the binary image 70, which accurately extracts the filipodia. In other words, the binarization method according to this embodiment is particularly suitable for generating the binary image 70 of the cell image 30 that include subcellular structures of the cells 90 (image elements of a high-frequency component locally existing in small areas) whose pixel values are likely to be low.

In this embodiment, the first threshold image 61a acquired by binarizing the normalized image 40 by using the first threshold 65, and the second threshold image 61b acquired by binarizing the normalized image 40 by using the second threshold 66 smaller than the first threshold 65 are included as the first image 61; and the step S7 of generating a binary image 70 of the cell image 31 includes a step S7a of removing a mismatch part of the second image 62 that does not match with the second threshold image 61b from the second image 62, and a step S7b of combining the first threshold image 61a with the second image 62 from which the mismatch part, which does not match with the second threshold image 61b, is removed. According to this configuration, noise except for the cells 90 in the second image 62 can be removed by removing the mismatch part, which does not match with the second threshold image 61b, from the second image 62. Also, because the first threshold image 61a is binarized by using the first threshold 65, which is higher than the second threshold 66, it is possible to prevent that the cellular images (white areas) extracted by the binarization include the background 93 and noise. The binary image 70 can be acquired to accurately extract the subcellular structures 92 of the cells 90 but prevent noise included in the binary image 70 by combining the second image 62a and the first threshold image 61a from which noise is removed. Consequently, for example, even if the original cell image 30 (cell image 31) is not of high quality, it is possible to generate a low-noise binary image 70.

### Modified Embodiments

Note that the embodiment disclosed this time must be considered as illustrative in all points and not restrictive. The scope of the present invention is not shown by the above description of the embodiments but by the scope of claims for patent, and all modifications (modified examples) within the meaning and scope equivalent to the scope of claims for patent are further included.

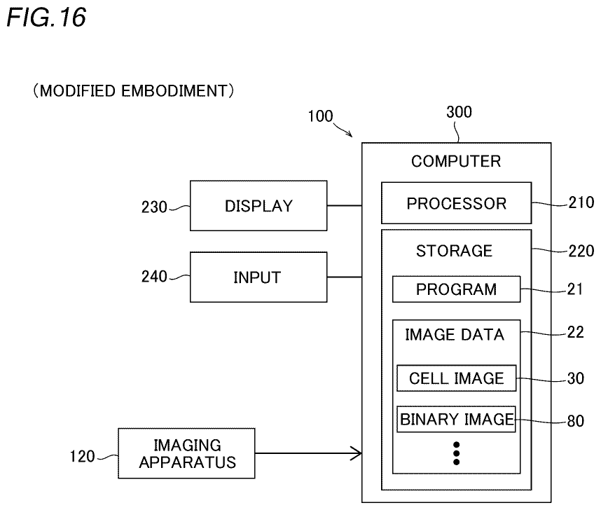

While the example in which the image processing apparatus 100 serves as a server in the image processing system 200 constructed of a client-server model has been shown in the aforementioned embodiment, the present invention is not limited to this. In the present invention, the image processing apparatus can be constructed of an independent computer, for example, as shown in FIG. 16. The image processing apparatus 100 shown in FIG. 16 includes a computer 300 including a processor 210 and a storage 220. The display 230 and an input 240 are connected to the computer 300. The computer 300 is connected to and can communicate with an imaging apparatus 120. The processor 210 of the computer 300 includes an image acquirer 11, a preprocessor 12, a background extractor 13, a relativizer 14, a frequency component extractor 15, a binarizer 16, a combiner 17, and a post-processor 18 as functional blocks shown in the foregoing embodiment (see FIG. 3)

While the example in which the single processor 10 (210) executes entire image processing (processes executed by the image acquirer 11, the preprocessor 12, the background extractor 13, the relativizer 14, the frequency component extractor 15, the binarizer 16, the combiner 17, and the post-processor 18) has been shown in the aforementioned embodiment and in the modified embodiment shown in FIG. 16, the present invention is not limited to this. A plurality of processors may share the processes applied to the cell image 30. Each process may be executed by one of the processors. The plurality of processors may be included in independent computers. In other words, the image processing apparatus 100 may be constructed of a plurality of computers for executing image processing.

While the example in which the background image 32 is generated by filtering the cell image 31 to remove the cells 90 in the cell image 31 has been shown in the aforementioned embodiment, the present invention is not limited to this. In the present invention, the background image 32 may be generated by a technique other than filtering. For example, the background image may be generated by applying Fourier transformation to the cell image 31, extracting a low frequency band corresponding to background brightness in the spatial frequency range and then applying inverse Fourier transformation to the image.

While the example in which the background image 32 representing the background brightness distribution is generated has been shown in the aforementioned embodiment, the present invention is not limited to this. In the present invention, the background brightness distribution may not be extracted as an image (background image 32). The background brightness distribution may, for example, be a function representing background brightness values (pixel values) at position coordinates in the image. In other words, the background brightness distribution may be expressed by a function of variables x and y coordinates of the image that provides pixel values corresponding to background brightness.

While the example in which the filtering the cell image 31 to remove the cells 90 in the cell image 31 is median filter has been shown in the aforementioned embodiment, the present invention is not limited to this. In the present invention, the filtering the cell image 31 to remove the cells 90 in the cell image 31 may be filtering other than median filter.

While the example in which filtering is applied to the reduced image 31a to remove the cells 90 from the reduced image in step S3 of extracting a background brightness distribution has been shown in the aforementioned embodiment, the present invention is not limited to this. In the present invention, filtering for removing the cells 90 may be applied to the cell image 31 having its original size without reducing the cell image 31. In this case, needless to say, a step of increasing the reduced background image 32a after filtering is unnecessary.

While the example in which pixel values of the normalized image 40 are determined by the equation of normalized image={(cell image 31b/background image 32)−1}×100(%) in step S4 of converting pixel values of pixels in the cell image 31 to relative values relative to the background image 32 has been shown in the aforementioned embodiment, the present invention is not limited to this. In the present invention, pixel values of the normalized image 40 may be determined by one other equation different from the aforementioned embodiment. Also, “1” may not be subtracted from a value that is obtained by dividing pixel values of the cell image 31b by pixel values of the background image 32.

While the example in which the frequency-component extraction image 50 is generated based on difference between the first smoothed image 41 and the second smoothed image 42 that have different frequency characteristics has been shown in the aforementioned embodiment, the present invention is not limited to this. In the present invention, the frequency-component extraction image 50 may be generated by a technique other than the technique using the difference between images. For example, the frequency-component extraction image may be generated by applying Fourier transformation to the normalized image 40, extracting a frequency band corresponding to the subcellular structures 92 in the spatial frequency range whereby removing other frequency components, and then applying inverse Fourier transformation to the image.

While the example in which a parameter set 57, which includes the first parameter and the second parameter, is selected from sets of predetermined parameters 57 to generate the frequency-component extraction image 50 (see FIG. 11) has been shown in the aforementioned embodiment, the present invention is not limited to this. In the present invention, a parameter set 57 may not be selected from sets of predetermined parameters 57 to generate the frequency-component extraction image 50. Inputs of the values of the first and second parameters may be separately accepted.

While the example in which smoothing the cell image to generate the first smoothed image 41 and the second smoothed image 42 is Gaussian filtering has been shown in the aforementioned embodiment, the present invention is not limited to this. Smoothing the cell image to generate the first smoothed image 41 and the second smoothed image 42 may be filtering other than Gaussian filtering. Other filtering can be, for example, moving average filtering. Here, smoothing parameters depend on types of filtering, that is, the smoothing parameters are not limited to the standard deviation σ.

While the example in which the subcellular structure 92 of the cell 90 is a filipodia of the cell 90 has been shown in the aforementioned embodiment, the present invention is not limited to this. The subcellular structure may be other small structure other than the filipodia. The subcellular structure of the cell is a small structure relative to the main structure of the cell in the cell image, and is a concept that depends on imaging magnification of the cell image. For example, in a case of a cell image of higher magnification than the cell image 30 (see FIG. 2), which s described in the aforementioned embodiment, including only a pseudopodium part of a cell, a part of the filipodia captured in the image can be defined as the subcellular structure.

While the example in which the first threshold image 61a and the second threshold image 61b that are generated by using different binarization thresholds are generated has been shown in the aforementioned embodiment, the present invention is not limited to this. In the present invention, only one first image 61 may be generated by using one binarization threshold. In this case, the first image 61 and the second image 62 are simply combined without executing step S7a.

While the example in which preprocessing for converting the color cell image 30 to a gray-scale cell image 31 is used has been shown in the aforementioned embodiment, the present invention is not limited to this. In a case in which the original cell image is a gray-scale image, the preprocessing is unnecessary. While the example in which a color component image that has the highest brightness value is selected to be converted into the gray-scale cell image 31 in the preprocessing has been shown in the aforementioned embodiment, pixel values of color component images may be averaged and then used to generate the gray-scale cell image 31.

While the example in which the post-processing is applied to the binary image 70 that is generated by combination so that the binary image 80 after post-processing is provided as a final processed image has been shown in the aforementioned embodiment, the present invention is not limited to this. In the present invention, the post-processing may not be applied. The binary image 70 that is generated by combination may be generated as the final processed image.

The specific values shown in the aforementioned embodiment such as kernel size, smoothing parameters, and binarization thresholds are illustratively shown, and they are not limited to the above values.

### Modes

The aforementioned exemplary embodiment will be understood as concrete examples of the following modes by those skilled in the art.

### Mode Item 1

An image processing method for binarization of a multi-value cell image includes a step of extracting a background brightness distribution in the cell image; a step of converting pixel values of pixels in the cell image into relative values relative to the background brightness distribution; a step of acquiring a frequency-component extraction image that extracts a predetermined frequency component corresponding to a subcellular structure of a cell from the converted cell image; a step to acquiring a first image by binarizing the converted cell image, and a second image by binarizing the frequency-component extraction image; and a step of generating a binary image of the cell image by combining the first image and the second image.

### Mode Item 2

In the image processing method according to mode item 1, a background image representing the background brightness distribution is generated by filtering the cell image to remove the cell in the cell image in the step of extracting a background brightness distribution.

### Mode Item 3

In the image processing method according to mode item 2, the filtering the cell image to remove the cell is median filter for a kernel size corresponding to a size of the cell in the cell image.

### Mode Item 4

In the image processing method according to mode item 2 or 3, the step of extracting a background brightness distribution includes a step of reducing the cell image, a step of filtering the reduced cell image to remove the cell in the reduced cell image, and a step of increasing the background image after being subjected to the step of filtering the reduced cell image to remove the cell back to a size of the cell image before being subjected to the step of reducing the cell image.

### Mode Item 5

In the image processing method according to any of mode items 2 to 4, the pixel values of the pixels in the cell image are converted into the relative values by dividing the pixel values of the pixels in the cell image by pixel values of pixels in the background image in the step of converting pixel values of pixels in the cell image into relative values relative to the background brightness distribution.

### Mode Item 6

In the image processing method according to any of mode items 1 to 5, the step of acquiring a frequency-component extraction image includes a step of generating a first smoothed image and a second smoothed image that have different frequency characteristics by smoothing the cell image, and a step of generating the frequency-component extraction image based on difference between the first smoothed image and the second smoothed image.

### Mode Item 7

In the image processing method according to mode item 6, the step of acquiring a frequency-component extraction image includes a step of selecting a parameter set, which includes a first parameter for generating the first smoothed image and a second parameter for generating the second smoothed image, from sets of predetermined parameters.

### Mode Item 8

In the image processing method according to mode item 7, the smoothing is Gaussian filtering; and parameters of the smoothing are standard deviations of the Gaussian filtering.

### Mode Item 9

In the image processing method according to any of mode items 1 to 8, the subcellular structure of the cell is a filipodia of the cell.

### Mode Item 10

In the image processing method according to any of mode items 1 to 9, the first image includes a first threshold image that is acquired by binarizing the cell image with a first threshold, and a second threshold image that is acquired by binarizing the cell image with a second threshold smaller than the first threshold; and the step of generating a binary image of the cell image includes a step of removing a mismatch part of the second image that does not match with the second threshold image from the second image, and a step of combining the first threshold image with the second image from which the mismatch part, which does not match with the second threshold image, is removed.

### Mode Item 11

An image processing apparatus includes an image acquirer that is configured to acquire a multi-value cell image; a background extractor that is configured to extract a background brightness distribution in the cell image; a relativizer that is configured to convert pixel values of pixels in the cell image into relative values relative to the background brightness distribution; a frequency component extractor that is configured to acquire a frequency-component extraction image that extracts a predetermined frequency component corresponding to a subcellular structure of a cell from the converted cell image; a binarizer that is configured to acquire a first image by binarizing the converted cell image by using binarization, and a second image by binarizing the frequency-component extraction image by using binarization; and a combiner that is configured to generate a binary image of the cell image by combining the first image and the second image.

### DESCRIPTION OF REFERENCE NUMERALS

11; image acquirer
13; background extractor
14; relativizer
15; frequency component extractor
16; binarizer
17; combiner
30, 31, 31b; cell image
31a; reduced image (reduced cell image)
32; background image
32a; reduced background image (filtered background image)
40; normalized image (relative-value-converted cell image)
41; first smoothed image
42; second smoothed image
50; frequency-component extraction image
57; parameter set
61; first image
61a; first threshold image
61b; second threshold image
62, 62a; second image
65; first threshold
66; second threshold
70, 80; binary image
90; cell
92; subcellular structure
93; background
100; image processing apparatus

1. An image processing method for binarization of a multi-value cell image, the method comprising:
a step of extracting a background brightness distribution in the cell image;
a step of converting pixel values of pixels in the cell image into relative values relative to the background brightness distribution;
a step of acquiring a frequency-component extraction image that extracts a predetermined frequency component corresponding to a subcellular structure of a cell from the converted cell image;
a step to acquiring a first image by binarizing the converted cell image, and a second image by binarizing the frequency-component extraction image; and
a step of generating a binary image of the cell image by combining the first image and the second image.

2. The image processing method according to claim 1, wherein a background image representing the background brightness distribution is generated by filtering the cell image to remove the cell in the cell image in the step of extracting a background brightness distribution.

3. The image processing method according to claim 2, wherein the filtering the cell image to remove the cell is median filter for a kernel size corresponding to a size of the cell in the cell image.

4. The image processing method according to claim 2, wherein
the step of extracting a background brightness distribution includes
a step of reducing the cell image,
a step of filtering the reduced cell image to remove the cell in the reduced cell image, and
a step of increasing the background image after being subjected to the step of filtering the reduced cell image to remove the cell back to a size of the cell image before being subjected to the step of reducing the cell image.

5. The image processing method according to claim 2, wherein the pixel values of the pixels in the cell image are converted into the relative values by dividing the pixel values of the pixels in the cell image by pixel values of pixels in the background image in the step of converting pixel values of pixels in the cell image into relative values relative to the background brightness distribution.

6. The image processing method according to claim 1, wherein
the step of acquiring a frequency-component extraction image includes
a step of generating a first smoothed image and a second smoothed image that have different frequency characteristics by smoothing the cell image, and
a step of generating the frequency-component extraction image based on difference between the first smoothed image and the second smoothed image.

7. The image processing method according to claim 6, wherein the step of acquiring a frequency-component extraction image includes a step of selecting a parameter set, which includes a first parameter for generating the first smoothed image and a second parameter for generating the second smoothed image, from sets of predetermined parameters.

8. The image processing method according to claim 7, wherein
the smoothing is Gaussian filtering; and
parameters of the smoothing are standard deviations of the Gaussian filtering.

9. The image processing method according to claim 1, wherein the subcellular structure of the cell is a filipodia of the cell.

10. The image processing method according to claim 1, wherein
the first image includes a first threshold image that is acquired by binarizing the cell image with a first threshold, and a second threshold image that is acquired by binarizing the cell image with a second threshold smaller than the first threshold; and
the step of generating a binary image of the cell image includes
a step of removing a mismatch part of the second image that does not match with the second threshold image from the second image, and
a step of combining the first threshold image with the second image from which the mismatch part, which does not match with the second threshold image, is removed.

11. An image processing apparatus comprising:
an image acquirer that is configured to acquire a multi-value cell image;
a background extractor that is configured to extract a background brightness distribution in the cell image;
a relativizer that is configured to convert pixel values of pixels in the cell image into relative values relative to the background brightness distribution;
a frequency component extractor that is configured to acquire a frequency-component extraction image that extracts a predetermined frequency component corresponding to a subcellular structure of a cell from the converted cell image;
a binarizer that is configured to acquire a first image by binarizing the converted cell image by using binarization, and a second image by binarizing the frequency-component extraction image by using binarization; and
a combiner that is configured to generate a binary image of the cell image by combining the first image and the second image.
